(19) United States
(12) Patent Application Publication
(10) Pub. No.: US 2024/0180515 A1
(43) Pub. Date: Jun. 6, 2024

(54) INFORMATION PROCESSING DEVICE AND NON-TRANSITORY COMPUTER READABLE STORAGE MEDIUM

(71) Applicant: NIHON KOHDEN CORPORATION, Tokyo (JP)

(72) Inventors: Hiraku Tabata, Tokorozawa-shi (JP); Takao Suzuki, Tokorozawa-shi (JP); Mitsuhiro Oura, Tokorozawa-shi (JP); Sou Kumagai, Tokorozawa-shi (JP)

(73) Assignee: NIHON KOHDEN CORPORATION, Tokyo (JP)

(21) Appl. No.: 18/517,711

(22) Filed: Nov. 22, 2023

(30) Foreign Application Priority Data
Dec. 2, 2022 (JP) 2022-193264

(51) Int. Cl. A61B 8/08; A61B 8/00

(52) U.S. Cl. CPC A61B 8/0891; A61B 8/085; A61B 8/4455; A61B 8/463; A61B 8/54

(57) ABSTRACT

An information processing device for processing ultrasonic wave information obtained from an ultrasonic wave sensor including a plurality of ultrasonic wave elements. The information processing device includes an obtainer configured to obtain the ultrasonic wave information from the ultrasonic wave sensor provided on a body surface of a subject, a recognizer configured to recognize a first blood vessel of the subject and a second blood vessel, based on the ultrasonic wave information, the second blood vessel being different from the first blood vessel, and a controller configured to cause a display to display comparison information in which first blood vessel information relating to the first blood vessel and second blood vessel information relating to the second blood vessel are compared with each other.

### CROSS-REFERENCE TO RELATED APPLICATIONS

This application is based on and claims priority under 35 USC 119 from Japanese Patent Application No. 2022-193264 filed on Dec. 2, 2022, the entire content of which is incorporated herein by reference.

### TECHNICAL FIELD

The presently disclosed subject matter relates to an information processing device for processing information obtained from an ultrasonic wave sensor including a plurality of ultrasonic wave elements, an information processing system including the information processing device, and a non-transitory computer readable storage medium.

### BACKGROUND ART

In recent years, the importance of diagnosis using an ultrasonic wave sensor including a plurality of ultrasonic wave elements has increased (see, for example, US 2020/0337680 A1). By using information obtained from the ultrasonic wave sensor, for example, a velocity (flow velocity) of blood and an amount (flow rate) of blood flowing through a blood vessel of a subject can be calculated.

It is desirable that states of a plurality of blood vessels can be compared using the information obtained from such an ultrasonic wave sensor. For example, by comparing the states of two blood vessels branched from one blood vessel, a doctor or the like can more accurately estimate states of an organ and a tissue connected to the blood vessels.

Therefore, an object of the presently disclosed subject matter is to provide an information processing device, an information processing system, and a non-transitory computer readable storage medium storing an information processing program capable of comparing states of a plurality of blood vessels.

### SUMMARY OF INVENTION

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to an aspect of the present disclosure, there is provided an information processing device for processing ultrasonic wave information obtained from an ultrasonic wave sensor including a plurality of ultrasonic wave elements, the information processing device including:
an obtainer configured to obtain the ultrasonic wave information from the ultrasonic wave sensor provided on a body surface of a subject;
a recognizer configured to recognize a first blood vessel of the subject and a second blood vessel, based on the ultrasonic wave information, the second blood vessel being different from the first blood vessel; and
a controller configured to cause a display to display comparison information in which first blood vessel information relating to the first blood vessel and second blood vessel information relating to the second blood vessel are compared with each other.

According to an aspect of the present disclosure, there is provided a non-transitory computer readable storage medium storing an information processing program for processing ultrasonic wave information obtained from an ultrasonic wave sensor including a plurality of ultrasonic wave elements,
the information processing program including instructions which, when executed by a computer, causes the computer to:
obtain the ultrasonic wave information from the ultrasonic wave sensor provided on a body surface of a subject;
recognize a first blood vessel of the subject and a second blood vessel, based on the ultrasonic wave information, the second blood vessel being different from the first blood vessel; and
cause a display to display comparison information in which first blood vessel information relating to the first blood vessel and second blood vessel information relating to the second blood vessel are compared with each other.

### BRIEF DESCRIPTION OF DRAWINGS

Exemplary embodiment(s) of the present invention will be described in detail based on the following figures, wherein:

FIG. 1 is a diagram of an example of a configuration of an information processing system according to an embodiment of the presently disclosed subject matter;

FIG. 2 is a diagram of an example of an attachment state of an ultrasonic wave sensor illustrated in FIG. 1;

FIG. 3A is a top view of the ultrasonic wave sensor illustrated in FIG. 1;

FIG. 3B is a side view of the ultrasonic wave sensor illustrated in FIG. 1;

FIG. 3C is a perspective view of the ultrasonic wave sensor illustrated in FIG. 1;

FIG. 4 is a block diagram of an example of a configuration of an information processing device illustrated in FIG. 1;

FIG. 5 is a block diagram of an example of a function of a CPU illustrated in FIG. 4;

FIG. 6 is a diagram of an example of comparison information displayed on an operation display illustrated in FIG. 4;

FIG. 7 is a diagram of a waveform at time t2 and a waveform at time t3 illustrated in FIG. 6 in an overlapping manner;

FIG. 8 is a diagram of another example of comparison information displayed on the operation display illustrated in FIG. 4;

FIG. 9 is a diagram of another example of comparison information displayed on the operation display illustrated in FIG. 4;

FIG. 10 is a diagram of another example of vital signs information displayed on the operation display together with the comparison information illustrated in FIG. 9;

FIG. 11 is a flowchart of an example of processing of the information processing device illustrated in FIG. 1;

FIG. 12 is a subroutine flowchart of step S105 illustrated in FIG. 11;

FIG. 13 is a diagram of an example of a relation between the ultrasonic wave sensor illustrated in FIG. 1 and a first blood vessel; and

FIG. 14 is an enlarged view of an XZ plane view illustrated in FIG. 13.

### DESCRIPTION OF EMBODIMENTS

Hereinafter, an ultrasonic wave diagnostic system (ultrasonic wave processing system) according to an embodiment of the presently disclosed subject matter will be described in detail with reference to the drawings. In the drawings, the same members are denoted by the same reference numerals. In addition, dimensional ratios in the drawings are exaggerated for convenience of the description, and may be different from actual ratios.

### EMBODIMENT

### (Configuration of Information Processing System)

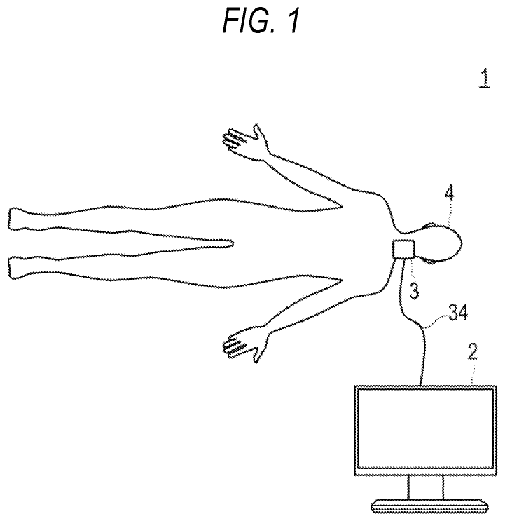

FIG. 1 is a schematic diagram of an example of a configuration of an information processing system 1 according to the present embodiment. The information processing system 1 is configured to display states of a plurality of blood vessels (first blood vessel 401 and second blood vessel 402 to be described later), using an ultrasonic wave sensor (ultrasonic wave sensor 3 to be described later), such that a user can compare the states of the plurality of blood vessels. The user is, for example, a healthcare worker such as a doctor and a nurse.

The information processing system 1 includes, for example, an information processing device 2, the ultrasonic wave sensor 3, and a cable 34. The cable 34 is configured to transmit an electrical signal between the ultrasonic wave sensor 3 and the information processing device 2. The cable 34 may be integrated with the ultrasonic wave sensor 3 or may be attachable to and detachable from the ultrasonic wave sensor 3. The cable 34 has, for example, a connector (not shown), and is connected to the information processing device 2 by inserting the connector into the information processing device 2. The ultrasonic wave sensor 3 is attached to a body surface of a subject 4. Information (ultrasonic wave information to be described later) obtained by the ultrasonic wave sensor 3 from the subject 4 is transmitted to the information processing device 2 via the cable 34. The information processing device 2 may be, for example, a patient monitor or a defibrillator configured to further obtain other vital signs data (blood pressure, oxygen saturation, respiration, and the like), and may be a computer such as a server or a PC.

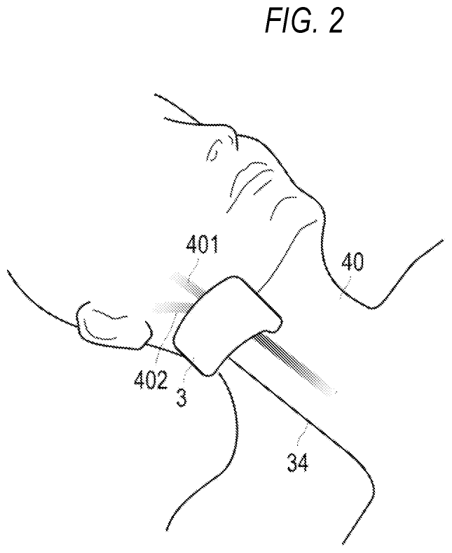

FIG. 2 illustrates an example of a configuration near the ultrasonic wave sensor 3 attached to the subject 4. The ultrasonic wave sensor 3 is used by being attached to the body surface of the subject 4, for example. For example, information relating to the first blood vessel 401 and the second blood vessel 402 is obtained by the ultrasonic wave sensor 3 attached to a cervical region 40 of the subject 4. For example, the first blood vessel 401 is an internal carotid artery, and the second blood vessel 402 is an external carotid artery. The ultrasonic wave sensor 3 is configured to measure the first blood vessel 401 and the second blood vessel 402 branched from a common carotid artery at a proximal position.

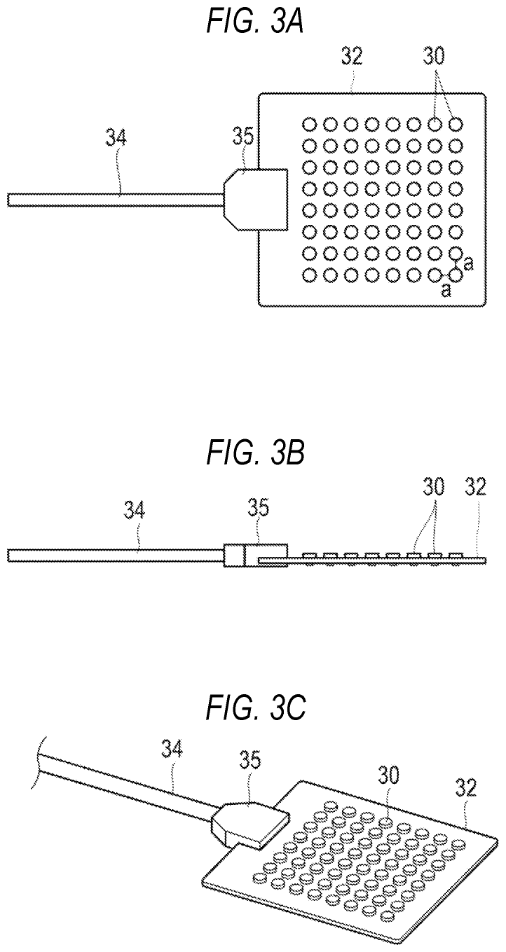

FIGS. 3A to 3C illustrate an example of a configuration of the ultrasonic wave sensor 3. FIG. 3A is a top view of the ultrasonic wave sensor 3, FIG. 3B is a side view of the ultrasonic wave sensor 3, and FIG. 3C is a perspective view of the ultrasonic wave sensor 3. The ultrasonic wave sensor 3 includes, for example, a plurality of ultrasonic wave elements 30, a sheet-shaped member 32 having flexibility, and a connection member 35.

The sheet-shaped member 32 has flexibility, and thus the sheet-shaped member 32 is deformed along a shape of an attached portion when attached to the body surface of the subject 4. The sheet-shaped member 32 has, for example, a quadrangular planar shape (FIG. 3A). The sheet-shaped member 32 is made of, for example, a resin material such as polyimide or silicon.

The plurality of ultrasonic wave elements 30 are, for example, embedded in a matrix in the sheet-shaped member 32. The ultrasonic wave elements 30 may be arranged on the sheet-shaped member 32. FIGS. 3A to 3C illustrate 64 ultrasonic wave elements 30 arranged, which is eight in a row direction and eight in a column direction, but the number and arrangement of the ultrasonic wave elements 30 are not limited thereto. For example, 96 ultrasonic wave elements may be arranged, which is 3 in the row direction and 32 in the column direction.

The plurality of ultrasonic wave elements 30 are arranged, for example, at equal intervals in the sheet-shaped member 32, and are arranged, for example, at intervals a in the row direction and the column direction. The interval between adjacent ultrasonic wave elements 30 may be known, and may be arranged such that the intervals in the row direction and the intervals in the column direction are different, or the intervals may be different depending on a position of the sheet-shaped member 32.

Each of the plurality of ultrasonic wave elements 30 includes, for example, a piezoelectric substance and an electrode. The ultrasonic wave element 30 is configured to transmit an ultrasonic wave in response to a transmission instruction transmitted from the information processing device 2 via the cable 34. In a case where the ultrasonic wave element 30 receives an ultrasonic wave (hereinafter, referred to as a reflected wave) reflected by a blood vessel, a bone, an organ, and the like in a body of the subject 4, a signal is transmitted from each of the ultrasonic wave elements 30 to the information processing device 2 via the cable 34.

The connection member 35 is configured to electrically connect the cable 34 and the plurality of ultrasonic wave elements 30, and is constituted by, for example, a wiring substrate.

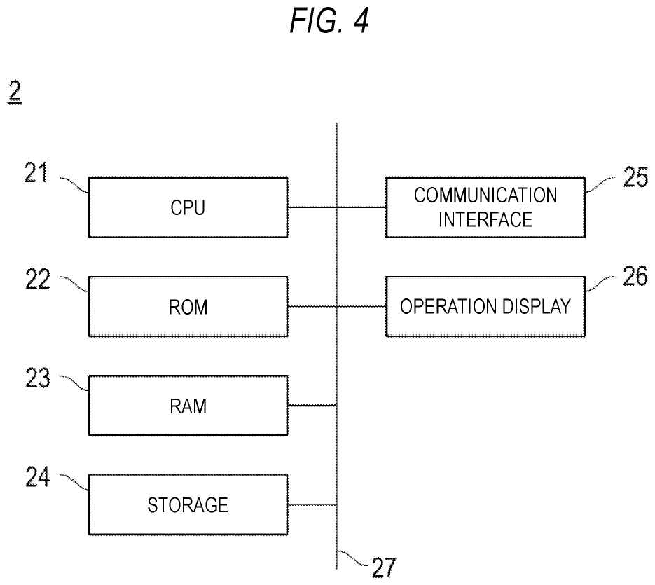

FIG. 4 is a block diagram of a schematic configuration of the information processing device 2. The information processing device 2 includes a central processing unit (CPU) 21, a read only memory (ROM) 22, a random access memory (RAM) 23, a storage 24, a communication interface 25, and an operation display 26. These components are communicably connected to one another via a bus 27.

The CPU 21 is configured to control the components and performs various types of arithmetic processing according to a program recorded in the ROM 22 or the storage 24. A specific function of the CPU 21 will be described later. The ROM 22 is configured to store various programs and various data. The RAM 23 is configured to temporarily store programs and data as a work area.

The storage 24 is configured to store various programs including an operating system and various data. For example, the storage 24 is installed with an application for transmitting and receiving various types of information to and from the ultrasonic wave sensor 3, and for determining a result (comparison information to be described later) to be output based on various types of information obtained from the ultrasonic wave sensor 3. In addition, the storage 24 is configured to store candidates of information to be output and information necessary for determining the result to be output based on various types of information. In a case where a machine learning model is used to determine the result to be output, training data or a learned model necessary for machine learning may be stored.

The communication interface 25 is an interface for communicating with other devices. A communication interface based on various wired or wireless standards is used as the communication interface 25. For example, the communication interface 25 is connected to the cable 34.

The operation display 26 is, for example, a touch panel display, displays various types of information, and receives various types of input from the user. Here, the operation display 26 corresponds to a specific example of a display of the presently disclosed subject matter. For example, the user can select one or more ultrasonic wave elements 30 from the plurality of ultrasonic wave elements 30, and can cause the selected ultrasonic wave elements 30 to transmit ultrasonic waves. The operation display 26 is configured to receive the selection of the ultrasonic wave elements 30. Information input by the user is transmitted to the CPU 21. The operation display 26 may be provided separately into an operator and the display. At this time, the operator includes, for example, an operation button, a mouse, or a keyboard, and the display includes a display or the like.

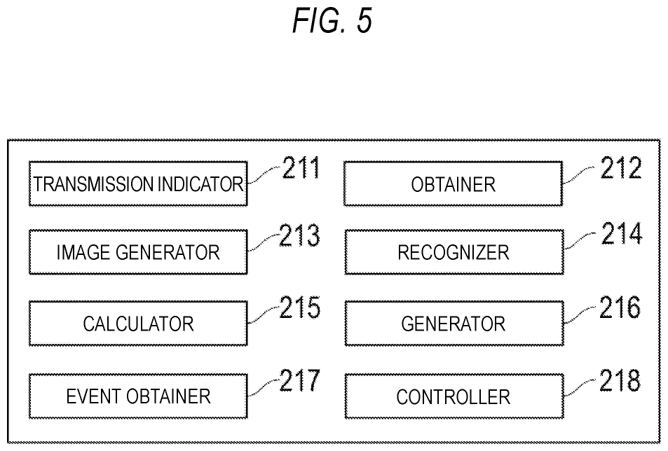

FIG. 5 is a block diagram of an example of a functional configuration of the CPU 21. In the information processing device 2, for example, when the CPU 21 reads the program stored in the storage 24 and executes processing to function as a transmission indicator 211, an obtainer 212, an image generator 213, a recognizer 214, a calculator 215, a generator 216, an event obtainer 217, and a controller 218.

For example, the transmission indicator 211 is configured to cause, to transmit ultrasonic waves, one or more ultrasonic wave elements 30 selected by the user on the operation display 26. For example, the transmission indicator 211 is configured to transmit a transmission instruction to the selected ultrasonic wave elements 30 via the cable 34 to drive the ultrasonic wave elements 30 and transmit the ultrasonic waves.

The obtainer 212 is configured to obtain ultrasonic wave information from the ultrasonic wave sensor 3. The ultrasonic wave information includes, for example, transmission information and reception information. The transmission information is information relating to the ultrasonic wave transmitted from the ultrasonic wave element 30, and includes, for example, information relating to positions of the driven ultrasonic wave elements 30, a driving voltage, a driving frequency, a waveform, a gain, a driving start time, a driving time, and the like of the ultrasonic wave element 30. The reception information is information relating to reflected waves reflected by a plurality of blood vessels (for example, the first blood vessel 401 and the second blood vessel 402) of the subject 4 and received by the plurality of ultrasonic wave elements 30, and includes, for example, information relating to a frequency, a waveform, intensity, a reception time, and the like of the reflected waves. In a case where an ultrasonic wave is transmitted from one ultrasonic wave element 30, the plurality of ultrasonic wave elements 30 receive reflected waves, for example.

The obtainer 212 is configured to obtain at least the reception information of the ultrasonic wave information from the ultrasonic wave sensor 3. For example, the obtainer 212 is configured to obtain the reception information from each of the plurality of ultrasonic wave elements 30 via the communication interface 125. The obtainer 212 may be configured to further obtain the reception information relating to the reflected wave reflected by the bone or the organ and received by the ultrasonic wave element 30. The obtainer 212 may be configured to obtain the transmission information of the ultrasonic wave information from the transmission indicator 211.

The image generator 213 is configured to generate image data of the plurality of blood vessels of the subject 4, based on the ultrasonic wave information obtained by the obtainer 212. The image generator 213 may be configured to generate image data using information directly obtained from the ultrasonic wave sensor 3. For example, the image generator 213 is configured to generate image data of an M-mode image, a B-mode image, a color Doppler-mode image, a pulse Doppler-mode image, or the like, by performing each processing on the ultrasonic wave information or the information obtained from the ultrasonic wave sensor 3.

The recognizer 214 is configured to recognize the plurality of blood vessels of the subject 4, based on the ultrasonic wave information obtained by the obtainer 212. The recognizer 214 is configured to recognize both the first blood vessel 401 and the second blood vessel 402 together with positions thereof. The recognizer 214 may be configured to label the first blood vessel 401 and the second blood vessel 402, using a pattern or the like of reflected waves of blood vessels stored in a position sensor or the storage 24. For example, the recognizer 214 is configured to label the first blood vessel 401 as an internal carotid artery, and the second blood vessel 402 as an external carotid artery. The recognizer 214 may be configured to recognize the first blood vessel 401 and the second blood vessel 402 from the image data generated by the image generator 213, or may be configured to recognize the first blood vessel 401 and the second blood vessel 402 according to an instruction from the user. The recognizer 214 may be configured to recognize positions of three or more blood vessels.

The calculator 215 is configured to calculate first blood vessel information relating to the first blood vessel 401 recognized by the recognizer 214, and second blood vessel information relating to the second blood vessel 402 recognized by the recognizer 214. The calculator 215 is configured to calculate the first blood vessel information and the second blood vessel information, based on the ultrasonic wave information obtained by the obtainer 212. The first blood vessel information is information relating to the state of the first blood vessel 401, and includes, for example, information relating to at least one of a velocity (flow velocity) and an amount (flow rate) of blood flowing through the first blood vessel 401. The second blood vessel information is information relating to the state of the second blood vessel 402, and includes, for example, information relating to at least one of a flow velocity and a flow rate of blood flowing through the second blood vessel 402. The first blood vessel information and the second blood vessel information may be flow velocities or flow rates of the first blood vessel 401 and the second blood vessel 402 or may be a processing result obtained by performing predetermined processing such as a statistical processing on the flow velocities or the flow rates of the first blood vessel 401 and the second blood vessel 402. The first blood vessel information and the second blood vessel information may include information relating to changes over time in the states of the first blood vessel 401 and the second blood vessel 402. In a case where the positions of three or more blood vessels are recognized by the recognizer 214, the calculator 215 may calculate blood vessel information relating to a part of the blood vessels selected by the user or may calculate blood vessel information relating to all of the blood vessels.

The generator 216 is configured to generate the comparison information in which the first blood vessel information and the second blood vessel information calculated by the calculator 215 are compared with each other. The comparison information is, for example, information in which the first blood vessel information and the second blood vessel information in the same time period are visually compared with each other. The details of the comparison information will be described later.

The event obtainer 217 is configured to obtain event information relating to an event occurring in the subject 4. The event information is information relating to an event that may affect the first blood vessel 401 and the second blood vessel 402 of the subject 4, and includes, for example, information relating to a content of the event and a time of the event. The event information includes, for example, information relating to a medication event to the subject 4. The information relating to the medication event includes information relating to at least one of a type, a dose, and an administration time of a drug administered to the subject 4. For example, the event obtainer 217 is configured to obtain the event information, based on the input from the user via the operation display 26. The event obtainer 217 may be configured to obtain event information from another device such as an electronic medical record.

The controller 218 is configured to cause the operation display 26 or the like to display the comparison information generated by the generator 216. As will be described in detail later, the user can easily compare the states of the first blood vessel 401 and the second blood vessel 402 visually, and can more accurately estimate states of an organ and a tissue connected to the first blood vessel 401 and the second blood vessel 402.

The comparison information includes, for example, at least one of a graph and a numerical value in which the first blood vessel information and the second blood vessel information in the same time period are compared with each other. The comparison information may include, for example, a diagram in which the first blood vessel information and the second blood vessel information in the same time period are compared with each other. The comparison information preferably includes information in which first blood vessel information and second blood vessel information in a first time period, and first blood vessel information and second blood vessel information in a second time period different from the first time period are compared with each other, and more preferably includes information in which the first blood vessel information and the second blood vessel information are compared with each other over time. Accordingly, the user can compare the states of the first blood vessel 401 with the second blood vessel 402 over time, and thus can more easily grasp changes in the states of the first blood vessel 401 and the second blood vessel 402.

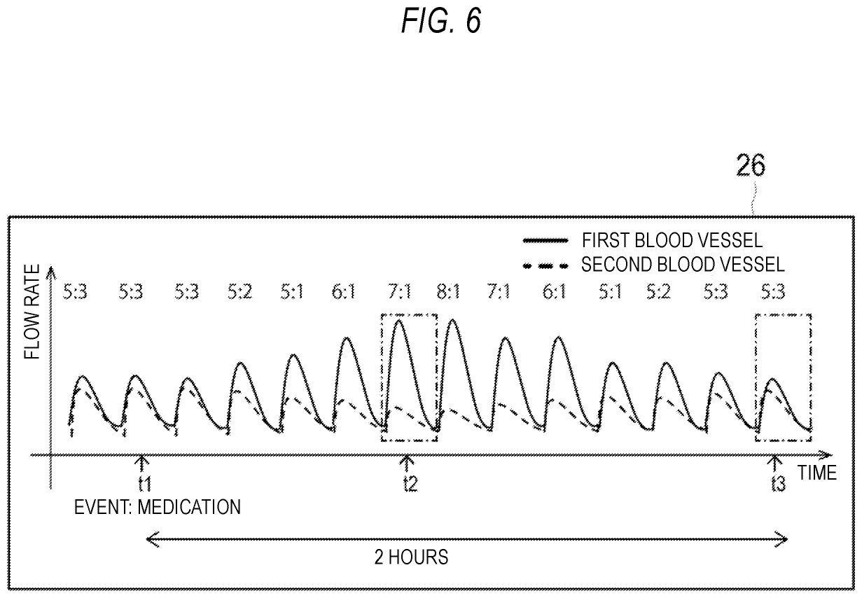

FIG. 6 illustrates an example of the comparison information displayed on the operation display 26. The comparison information includes, for example, a graph representing changes over time in the first blood vessel information and the second blood vessel information side by side. FIG. 6 illustrates waveforms representing changes over time in the flow rates of the first blood vessel 401 and the second blood vessel 402. In FIG. 6, a vertical axis represents the flow rates of the first blood vessel 401 and the second blood vessel 402, and a horizontal axis represents time. In FIG. 6, a solid line represents the flow rate of the first blood vessel 401, and a broken line represents the flow rate of the second blood vessel 402.

The comparison information may include a numerical value in which the changes over time in the first blood vessel information and the second blood vessel information are compared with each other. In FIG. 6, a numerical value (for example, 5:3 of time t1) representing an amplitude ratio of the waveforms of the first blood vessel 401 and the second blood vessel 402 is expressed at a predetermined time interval.

The controller 218 may be configured to cause the operation display 26 to display, in an overlapping manner, the first blood vessel information and the second blood vessel information in the first time period, and the first blood vessel information and the second blood vessel information in the second time period.

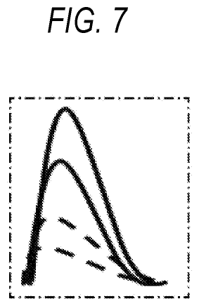

FIG. 7 illustrates waveforms of the first blood vessel 401 and the second blood vessel 402 in the vicinity of a time t2 in FIG. 6, and waveforms of the first blood vessel 401 and the second blood vessel 402 in the vicinity of a time t3 in FIG. 6. For example, the controller 218 is configured to cause the operation display 26 to display the first blood vessel information and the second blood vessel information in a plurality of time periods selected by the user.

For example, the controller 218 may be configured to cause the operation display 26 to display the event information together with the comparison information. FIG. 6 illustrates a medication event at the time t1. It is preferred that the controller 218 is configured to cause the operation display 26 to display a related time of an event (for example, time t1) and measurement times of the first blood vessel information and the second blood vessel information in association with each other. Accordingly, the user can more easily grasp the influence of the event on the first blood vessel information and the second blood vessel information. The controller 218 may be configured to cause the operation display 26 to display event information including a type, a dose, and the like of the drug administered to the subject 4.

The controller 218 may be configured to cause the operation display 26 to display information on the event. For example, the controller 218 may be configured to cause the operation display 26 to display a time until states of the first blood vessel 401 and the second blood vessel 402 changed due to the event return to the states before the event. In FIG. 6, the flow rate of the first blood vessel 401 starts to increase and the flow rate of the second blood vessel 402 starts to decrease, due to the medication event at the time t1. Thereafter, in the vicinity of the time t2, the increase in the flow rate of the first blood vessel 401 and the decrease in the flow rate of the second blood vessel 402 stop, and the decrease in the flow rate of the first blood vessel 401 and the increase in the flow rate of the second blood vessel 402 start. At the time t3, the flow rate of the first blood vessel 401 and the flow rate of the second blood vessel 402 return to approximately the states at the time t1. At this time, for example, the controller 218 may be configured to cause the operation display 26 to display a time (2 hours) from the time t1 to the time t3.

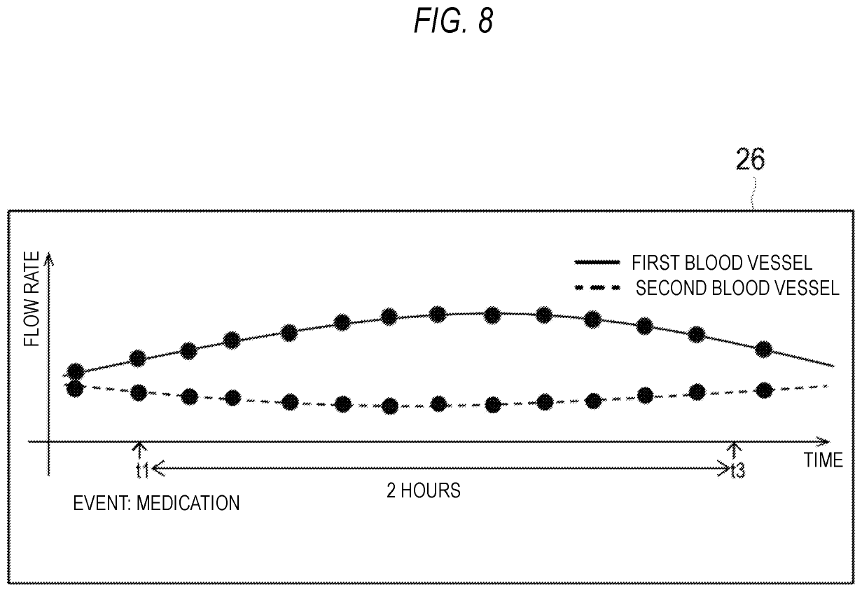

FIG. 8 illustrates another example of the comparison information displayed on the operation display 26. The comparison information may include a graph representing side-by-side changes over time in processing results obtained by performing predetermined processing on the flow rates of the first blood vessel 401 and the second blood vessel 402. FIG. 8 illustrates side-by-side curves representing changes over time in average values of the flow rates of the first blood vessel 401 and the second blood vessel 402 calculated at a predetermined time interval. In FIG. 8, a vertical axis represents the average value of each of the flow rates of the first blood vessel 401 and the second blood vessel 402, and a horizontal axis represents time. In FIG. 8, a solid line represents the average value of the flow rates of the first blood vessel 401, and a broken line represents the average value of the flow rates of the second blood vessel 402. The controller 218 may be configured to display changes over time of maximum values of the flow rates of the first blood vessel 401 and the second blood vessel 402 in one beat side by side. The controller 218 may be configured to display envelopes of the waveforms (see FIG. 6) representing the changes over time in the flow rates of the first blood vessel 401 and the second blood vessel 402 side by side. The operation display 26 may be configured to display a numerical value indicating average values or the like of the flow rates of the first blood vessel 401 and the second blood vessel 402 for each time.

The controller 218 may be configured to further cause the operation display 26 to display information synthesized by performing a fitting processing on the curves (see FIG. 8) representing the changes over time in the flow rates of the first blood vessel 401 and the second blood vessel 402. The fitting processing is, for example, an extreme value distribution, a normal distribution, or a quadratic function. For example, the controller 218 is configured to cause the operation display 26 to display a Q value and a half width of the curve representing the change over time in the flow rate of each of the first blood vessel 401 and the second blood vessel 402, a value representing the sharpness of the curve, or the like. In other words, the first blood vessel information and the second blood vessel information may include information relating to the curves representing the changes over time in the flow velocity or the flow rate.

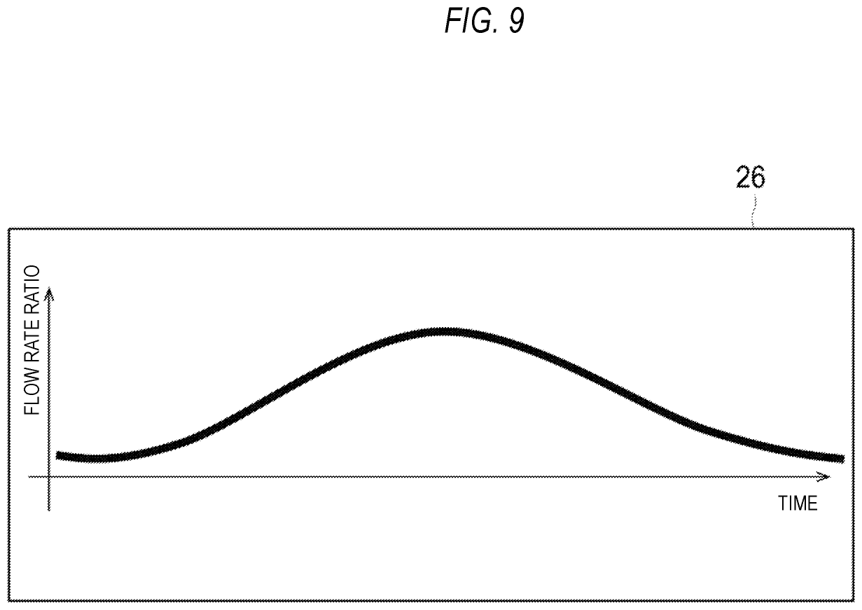

FIG. 9 illustrates another example of the comparison information displayed on the operation display 26. The comparison information may include at least one of a graph and a numerical value representing integrated information obtained by integrating the first blood vessel information and the second blood vessel information in the same time period. FIG. 9 is a graph representing a change over time in a ratio of the flow rates of the first blood vessel 401 and the second blood vessel 402 (flow rate of the first blood vessel 401 to flow rate of the second blood vessel 402) in the same time period. In FIG. 9, a vertical axis represents the ratio of the flow rates of the first blood vessel 401 and the second blood vessel 402, and a horizontal axis represents time.

The controller 218 may be configured to further cause the operation display 26 to display information obtained by performing a fitting processing on the curve (see FIG. 9) representing the change over time in the integrated information. The fitting processing is, for example, an extreme value distribution, a normal distribution, or a quadratic function. For example, the controller 218 is configured to cause the operation display 26 to display a Q value and a half width of the curve representing the change over time in the integrated information, a value representing the sharpness of the curve, or the like. In other words, the comparison information may include information relating to the curve representing the change over time in the integrated information.

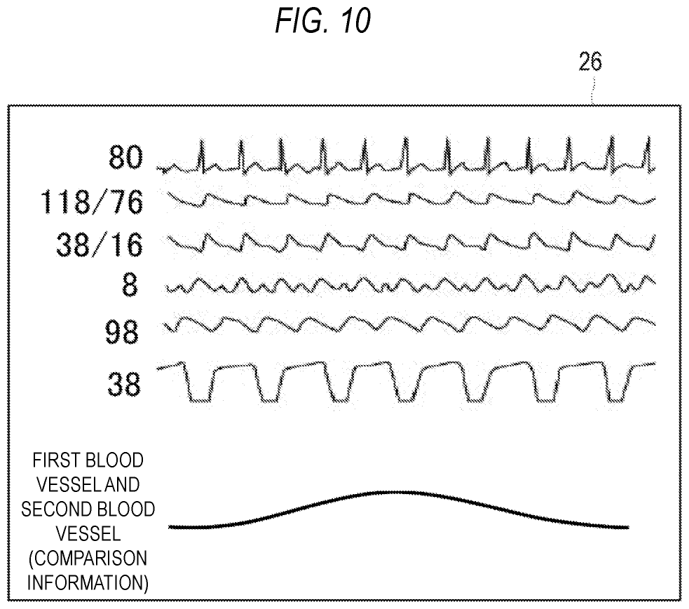

FIG. 10 illustrates the comparison information displayed on the operation display 26 together with other vital signs information. The controller 218 may be configured to cause the operation display 26 to display the comparison information together with the other vital signs information such as a heart rate, an arterial blood pressure, and SpO2 measured for the subject 4. Accordingly, the user can further easily compare the states of the first blood vessel 401 and the second blood vessel 402 with the other vital signs information. The controller 218 may be configured to cause a display other than the operation display 26 to display the comparison information, for example, a display such as a bed-side monitor or a central monitor to display the comparison information.

### (Processing Method of Information Processing Device)

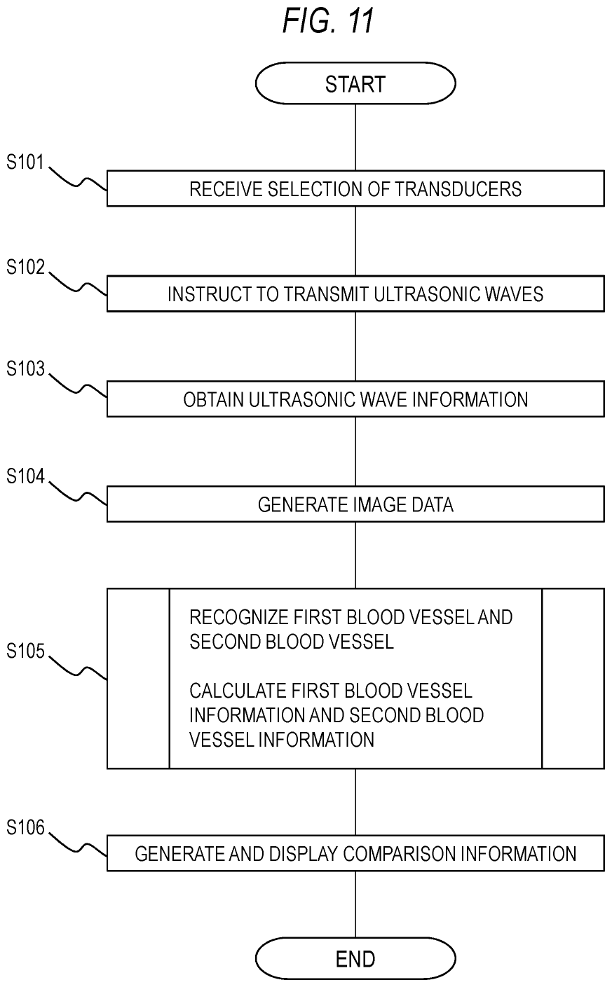

FIG. 11 is a flowchart of an example of processing performed by the information processing device 2. The flowchart can be executed according to a program stored in the information processing device 2. For example, the information processing device 2 is configured to obtain information from the ultrasonic wave sensor 3 attached to the cervical region 40 of the subject 4.

First, the information processing device 2 is configured to receive the selection of the ultrasonic wave elements 30 configured to transmit ultrasonic waves (step S101). For example, when the user inputs the selection of the ultrasonic wave elements 30 to the operation display 26, the information processing device 2 receives the selection of the ultrasonic wave elements 30. For example, the user can select the ultrasonic wave elements 30 arranged in the vicinity of the first blood vessel 401 and the second blood vessel 402 while confirming images of the first blood vessel 401 and the second blood vessel 402 displayed on the operation display 126.

Next, the information processing device 2 instructs the selected ultrasonic wave elements 30 to transmit the ultrasonic waves (step S102). For example, the information processing device 2 transmits a transmission instruction to the selected ultrasonic wave elements 30 via the communication interface 25 to transmit the ultrasonic waves to the ultrasonic wave elements 30.

Subsequently, the information processing device 2 obtains transmission information and reception information (step S103). Next, the information processing device 2 generates image data of the first blood vessel 401 and the second blood vessel 402, based on the information obtained from the ultrasonic wave sensor 3 (step S104).

Thereafter, the information processing device 2 recognizes positions of the first blood vessel 401 and the second blood vessel 402, and calculates the first blood vessel information relating to the first blood vessel 401 and the second blood vessel information relating to the second blood vessel 402 (step S105). The step will be described in detail later. The information processing device 2 may be configured to perform the processing of step S105 simultaneously with the processing of step S104, or may perform the processing of step S105 before the processing of step S104.

Thereafter, the information processing device 2 generates comparison information based on the first blood vessel information and the second blood vessel information calculated in step S105, displays the comparison information on the operation display 26 or the like (step S106), and ends the processing.

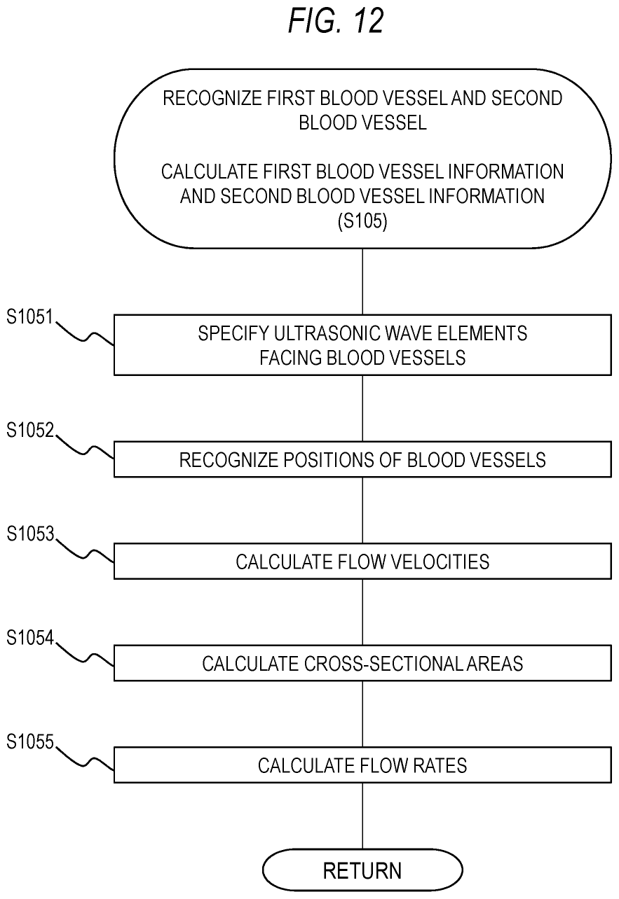

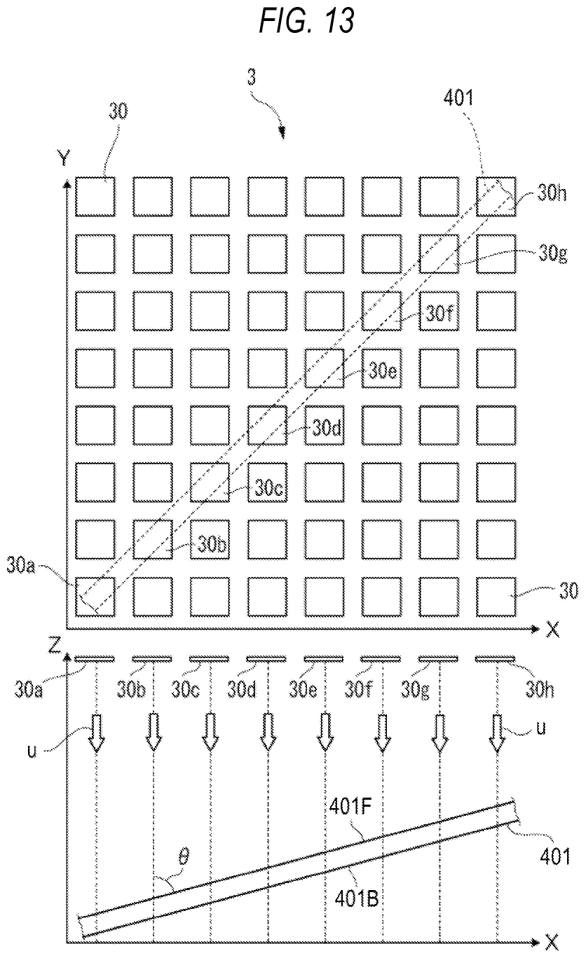

Here, the processing of step S105 will be described with reference to FIGS. 12 and 13. FIG. 12 is a subroutine flowchart of step S105, and FIG. 13 illustrates an example of a relation between the first blood vessel 401 and the ultrasonic wave sensor 3 (ultrasonic wave elements 30). Hereinafter, arrangement directions of the ultrasonic wave elements 30 may be referred to as an X direction and a Y direction, and a direction orthogonal to the X direction and the Y direction may be referred to as a Z direction. The ultrasonic wave element 30 is configured to transmit an ultrasonic wave u in a direction intersecting the X direction and the Y direction.

The information processing device 2 specifies the ultrasonic wave elements 30 facing the first blood vessel 401 and the second blood vessel 402 in the Z direction, based on the ultrasonic wave information obtained in the processing of step S103 (FIG. 11) (step S1051). For example, the ultrasonic wave elements 30a to 30h among the plurality of ultrasonic wave elements 30 face the first blood vessel 401 (FIG. 13). For example, the information processing device 2 specifies the ultrasonic wave elements 30a to 30h facing the first blood vessel 401 as follows.

The ultrasonic wave (ultrasonic wave u) is reflected at a boundary between media having different acoustic impedances, and thus is reflected at a boundary between the first blood vessel 401 and a physiological tissue adjacent to the first blood vessel 401. In particular, a difference in acoustic impedances between the first blood vessel 401 and the physiological tissue adjacent to the first blood vessel 401 is large, and thus the ultrasonic wave u is strongly reflected on a front wall 401F and a rear wall 401B of the first blood vessel 401. The information processing device 2 can specify the ultrasonic wave elements 30a to 30h due to the strong reflection of the ultrasonic wave u.

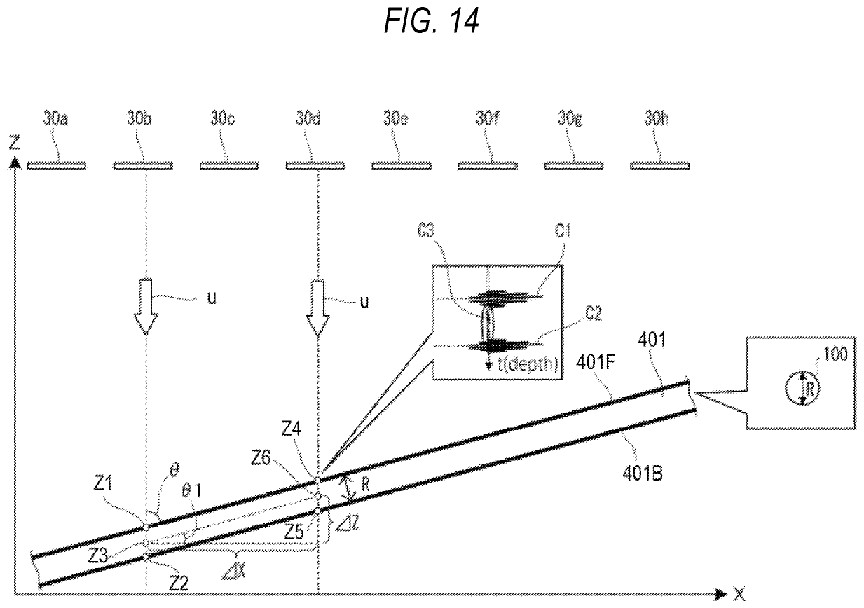

Alternatively, a pattern of a reflected wave of the ultrasonic wave u reflected by the front wall 401F and the rear wall 401B of the first blood vessel 401 may be known. The information processing device 2 may specify the ultrasonic wave elements 30 (ultrasonic wave elements 30a to 30h) that obtain predetermined reception information (for example, reflected waves C1 and C2 illustrated in FIG. 14 to be described later).

Same or similarly, the information processing device 2 specifies the ultrasonic wave elements 30 facing the second blood vessel 402. Next, the information processing device 2 recognizes the positions of the first blood vessel 401 and the second blood vessel 402 (step S1052).

FIG. 14 illustrates an example of a relation between the ultrasonic wave elements 30a to 30h and the first blood vessel 401 in the X direction and the Z direction. For example, the information processing device 2 recognizes the position of the first blood vessel 401 by determining positions Z1 to Z6 of the first blood vessel 401 facing the two ultrasonic wave elements 30 (for example, the ultrasonic wave elements 30b and 30d) among the ultrasonic wave elements 30a to 30h.

The positions Z1 and Z2 are positions in the Z direction of the front wall 401F and the rear wall 401B of the first blood vessel 401 facing the ultrasonic wave element 30b, respectively, and the position Z3 is an intermediate position between the position Z1 and the position Z2. The positions Z4 and Z5 are positions in the Z direction of the front wall 401F and the rear wall 401B of the first blood vessel 401 facing the ultrasonic wave element 30d, respectively, and the position Z6 is an intermediate position between the position Z4 and the position Z5.

For example, the information processing device 2 can determine the position Z1 as follows. The information processing device 2 specifies, based on the obtained ultrasonic wave information, a time (time t0) at which the ultrasonic wave u is transmitted from the ultrasonic wave element 30b to the first blood vessel 401, and a time (time t4) at which the ultrasonic wave u is reflected by the front wall 401F and received by the ultrasonic wave element 30b. Next, the information processing device 2 determines a distance L1 in the Z direction between the ultrasonic wave element 30b and the front wall 401F, based on the times t0 and t4. The distance L1 can be determined according to the following expression (1). The information processing device 2 determines the position Z1, based on the distance L1 between the ultrasonic wave element 30b and the front wall 401F.

L1=c(t4−t0)/2 (1)

c is a sound speed of the ultrasonic wave u traveling in the physiological tissue.

The information processing device 2 is configured to determine the position Z2, in the same manner as or similarly to the position Z1. Specifically, the information processing device 2 first specifies, based on the obtained ultrasonic wave information, the time (time t0) at which the ultrasonic wave u is transmitted from the ultrasonic wave element 30b to the first blood vessel 401, and a time (time t5) at which the ultrasonic wave u is reflected by the rear wall 401B and received by the ultrasonic wave element 30b. Next, the information processing device 2 determines a distance L2 in the Z direction between the ultrasonic wave element 30b and the rear wall 401B, based on the times t0 and t5. The distance L2 can be determined according to the following expression (2). The information processing device 2 determines the position Z2, based on the distance L2 between the ultrasonic wave element 30b and the rear wall 401B.

L2=c(t5−t0)/2 (2)

c is a sound speed of the ultrasonic wave u traveling in the physiological tissue.

After determining the positions Z1 and Z2, the information processing device 2 determines the position Z3, which is the intermediate position between the position Z1 and the position Z2, according to the following expression (3).

Z1=(Z1+Z2)/2 (3)

In the same manner as or similarly to the positions Z1 to Z3, the information processing device 2 determines the positions Z4 to Z6, in the Z direction, facing the ultrasonic wave element 30d and recognizes the position of the first blood vessel 401. The information processing device 2 recognizes the position of the second blood vessel 402, in the same manner as or similarly to the first blood vessel 401.

After recognizing the positions of the first blood vessel 401 and the second blood vessel 402, the information processing device 2 calculates flow velocities of the first blood vessel 401 and the second blood vessel 402 (step S1053). The information processing device 2 calculates the flow velocities of the first blood vessel 401 and the second blood vessel 402, for example, using a Doppler shift frequency and an angle formed by each of the first blood vessel 401 and the second blood vessel 402 with respect to the Z direction.

For example, the information processing device 2 determines an angle θ formed by the first blood vessel 401 with respect to the Z direction as follows. The information processing device 2 first determines an angle (angle θ1) formed by the first blood vessel 401 with respect to the X direction, based on a distance ΔZ in the Z direction between the position Z6 and the position Z3 and a distance ΔX in the X direction between the ultrasonic wave element 30b and the ultrasonic wave element 30d. The angle θ1 is determined according to, for example, the following expression (4).

θ1=tan⁻¹(ΔZ/ΔX) (4)

Here, the angle θ and the angle θ1 have a relation expressed according to the following expression (5). Accordingly, the information processing device 2 can determine the angle θ.

θ=(90°−θ1) (5)

Same or similarly, the information processing device 2 can determine the angle formed by the second blood vessel 402 with respect to the Z direction.

After determining the angle formed by each of the first blood vessel 401 and the second blood vessel 402 with respect to the Z direction, the information processing device 2 calculates the Doppler shift frequency in each of the first blood vessel 401 and the second blood vessel 402.

In a case where the velocity (flow velocity) of the blood flowing through the first blood vessel 401 is v, erythrocytes contained in the blood move through the first blood vessel 401 at the velocity v. The erythrocytes are observed to be moving in the Z direction at a speed of v cos θ, and a frequency change due to a Doppler effect occurs in the ultrasonic wave u reflected by the erythrocytes.

In a case where a frequency of the ultrasonic wave u transmitted from the ultrasonic wave element 30 is fₒ, the Doppler shift frequency is f_{d}, the velocity of the blood flowing through the first blood vessel 401 is v, and the sound speed of the ultrasonic wave u traveling in the physiological tissue is c, the Doppler shift frequency f_{d} is obtained according to the following expression (6).

f_{d}=2vfₒ×cos θ/c (6)

According to the expression (6), it is understood that the Doppler shift frequency f_{d} changes in proportion to the velocity v of the blood flowing through the first blood vessel 401. In other words, the velocity v (flow velocity) can be measured by measuring the Doppler shift frequency f_{d}.

The information processing device 2 can calculate the Doppler shift frequency f_{d}, using, for example, a pulse Doppler method or a color Doppler method. The Doppler shift frequency f_{d} is calculated using the pulse Doppler method, for example, as follows. The information processing device 2 first obtains data indicating a change in a sum of luminance values of a reflected wave C3 (see FIG. 14) of the ultrasonic wave u reflected by the erythrocytes in the first blood vessel 401. Thereafter, the information processing device 2 calculates the Doppler shift frequency f_{d} by performing a fast Fourier transform (FFT) processing on the obtained data.

In a case where the color Doppler method is used, the information processing device 2 calculates the Doppler shift frequency f_{d} by using, for example, an autocorrelation technique. In a case where the frequency of the ultrasonic wave is 8 kHz (in other words, in a case where 8000 ultrasonic wave pulses are output per second), the information processing device 2 may calculate the Doppler shift frequency f_{d} for every 140 ultrasonic wave pulses. At this time, an update rate of the Doppler shift frequency f_{d} is 57 Hz.

In the pulse Doppler method, a calculation load by FFT is relatively large. Therefore, it is preferred to use the color Doppler method having a relatively small calculation load, for the calculation of the Doppler shift frequency f_{d}.

The information processing device 2 calculates the velocity v (flow velocity) of the blood flowing through the first blood vessel 401 according to the expression (6), using the angle θ and the Doppler shift frequency f_{d} obtained as described above. Same or similarly, the information processing device 2 calculates the flow velocity of the second blood vessel 402.

After calculating the flow velocities of the first blood vessel 401 and the second blood vessel 402, the information processing device 2 calculates cross-sectional areas of the first blood vessel 401 and the second blood vessel 402 (step S1054). The cross-sectional area is a cross-sectional area perpendicular to an axial direction of each of the first blood vessel 401 and the second blood vessel 402, and indicates a cross-sectional area of a hollow portion of each of the first blood vessel 401 and the second blood vessel 402 through which blood flows.

For example, a cross-sectional area S of the first blood vessel 401 is obtained according to the following expression (7), using an inner diameter R of the first blood vessel 401.

S=πR²/4 (7)

A distance between the position Z1 and the position Z2 is |Z1−Z2|, and thus the inner diameter R is expressed according to the following expression (8). The inner diameter R may be calculated using a distance |Z3−Z4| between the position Z3 and the position Z4.

R=|Z1−Z2|sin θ (8)

The information processing device 2 calculates the cross-sectional area S of the first blood vessel 401 according to, for example, the expression (7) and the expression (8). Same or similarly, the information processing device 2 calculates the cross-sectional area of the second blood vessel 402.

After calculating the cross-sectional areas of the first blood vessel 401 and the second blood vessel 402, the information processing device 2 calculates the flow rates of the first blood vessel 401 and the second blood vessel 402. A flow rate Q of the first blood vessel 401 can be calculated according to the following expression (9), using, for example, the velocity v and the cross-sectional area S of the blood flowing through the first blood vessel 401. Same or similarly, the information processing device 2 calculates the flow rate of the second blood vessel 402.

Q=Sv (9)

After recognizing the positions of the first blood vessel 401 and the second blood vessel 402 in this manner, the information processing device 2 calculates the first blood vessel information and the second blood vessel information including the flow velocity and the flow rate of each of the first blood vessel 401 and the second blood vessel 402.

### (Action and Effect of Information Processing Device and Information Processing System)

In the information processing device 2 and the information processing system 1 according to the present embodiment, the operation display 26 is configured to display the comparison information in which the first blood vessel information relating to the first blood vessel 401 and the second blood vessel information relating to the second blood vessel 402 are compared with each other. Therefore, the user can compare the states of the first blood vessel 401 and the second blood vessel 402, and more accurately estimate the forms of the organ and tissue connected to the blood vessels. The action and effect will be described below.

A common carotid artery branches into an internal carotid artery and an external carotid artery, the internal carotid artery is responsible for cerebral circulation, and the external carotid artery is responsible for blood flows to face and scalp. A vasoactive agonist is often administered to a patient who suffers from circulatory disorder in the brain such as head damage and post-cardiac arrest syndrome (PCAS) in order to ensure an amount of circulating blood in the brain. In a case where a peripheral vascular contractor is administered to the patient, a blood pressure increases with an increase in vascular resistance, and thus an increase in the blood flow of the internal carotid artery can be expected. However, the blood flow in the internal carotid artery depends on a ratio of a blood vessel resistance of the internal carotid artery to a blood vessel resistance of the external carotid artery, and thus it may be difficult to determine whether an appropriate treatment can be performed even if the blood flow in only one of the arteries is confirmed.

Regarding this, in the information processing system 1 and the information processing device 2, the operation display 26 is configured to display the comparison information in which the first blood vessel information relating to the first blood vessel 401 and the second blood vessel information relating to the second blood vessel 402 are compared with each other. For example, the changes over time in the flow velocities or the flow rates of the first blood vessel 401 and the second blood vessel 402 are displayed (for example, FIG. 6). Therefore, the user can relatively compare the states of the first blood vessel 401 and the second blood vessel 402, and more accurately estimate the states of the organ and tissue connected to the first blood vessel 401 and the second blood vessel 402. Therefore, based on the first blood vessel 401, the second blood vessel 402, and the states of the organ and tissue connected to the first blood vessel 401 and the second blood vessel 402, the user can more accurately select a drug to be administered to the subject 4, and make a medication plan or the like.

In particular, the information relating to the first blood vessel 401 and the second blood vessel 402 branched from a third blood vessel at a proximal position, such as the internal carotid artery and the external carotid artery branched from the common carotid artery, often includes important information for measuring effects of medication or the like. Therefore, the first blood vessel 401 and the second blood vessel 402 to be compared are preferably branched from the third blood vessel at the proximal position. For example, the first blood vessel 401 and the second blood vessel 402 may be a left renal artery and a right renal artery branched from an abdominal aorta. By comparing the states of the first blood vessel 401 and the second blood vessel 402, it is possible to estimate, for example, a failure state or the like of one of the kidneys. Alternatively, the first blood vessel 401 and the second blood vessel 402 may be a right common iliac artery and a left common iliac artery branched from an abdominal aorta. By comparing the states of the first blood vessel 401 and the second blood vessel 402, it is possible to estimate, for example, thrombus or the like of one of the blood vessels.

The comparison information displayed on the operation display 26 preferably includes information in which the first blood vessel information and the second blood vessel information are compared with each other over time, and more preferably includes the first blood vessel information and the second blood vessel information before and after the medication event to the subject 4. Accordingly, the user can easily confirm the influence on the first blood vessel 401 and the second blood vessel 402 due to medication to the subject 4.

It is preferred that the controller 218 is configured to cause the operation display 26 to display the event information relating to the medication event or the like together with the comparison information. Accordingly, the user can easily confirm a relation between the event and the comparison information.

As described above, the information processing device and the information processing system of the presently disclosed subject matter have been described in the embodiment. However, it is needless to say that the presently disclosed subject matter can be appropriately added, modified, or omitted by those skilled in the art within the scope of the technical idea thereof.

For example, although FIGS. 6 to 9 illustrate an example in which the operation display 26 displays the comparison information in which the flow rates of the first blood vessel 401 and the second blood vessel 402 are compared with each other, the operation display 26 may display the comparison information in which the flow velocities of the first blood vessel 401 and the second blood vessel 402 are compared with each other, or may display the comparison information in which both the flow rates and the flow velocities are compared with each other.

The first blood vessel information and the second blood vessel information may be information indicating the states of the first blood vessel 401 and the second blood vessel 402, and may be information other than the flow velocities or the flow rates.

In the embodiment, the example is described in which the operation display 26 displays the comparison information in which the first blood vessel information and the second blood vessel information of two blood vessels (the first blood vessel 401 and the second blood vessel 402) are compared with each other, but the operation display 26 may display comparison information in which blood vessel information of three or more blood vessels are compared with each other.

In the embodiment, an example is described in which the flow velocity and the flow rate of the first blood vessel 401 are calculated using the angle θ and the Doppler shift frequency f_{d}, and the flow velocity and the flow rate of the first blood vessel 401 and the second blood vessel 402, may be calculated by another method.

In the embodiment, an example is mainly described in which the event occurring in the subject 4 is the medication event, but the event occurring in the subject 4 may be another treatment event such as surgery, or may be an event in daily life such as eating and drinking and taking a bath.

In the embodiment, the example is described in which the ultrasonic wave sensor 3 is used by being attached to the cervical region 40 of the subject 4, and the ultrasonic wave sensor 3 may be used by being attached to other parts of the subject 4. In addition, the ultrasonic wave sensor 3 may be used without being attached.

In the embodiment, an example is described in which the ultrasonic wave elements 30 are provided at the constant interval (interval a) in the ultrasonic wave sensor 3, but the intervals between adjacent ultrasonic wave elements 30 may be different depending on the position of the ultrasonic wave sensor 3.

In the embodiment, an example is described in which the information processing device 2 outputs image data of a blood vessel or the like of the subject 4, and the information processing device 2 may not output the image data.

The means and method for performing various types of processing in the information processing device 2 according to the embodiment can be implemented by a dedicated hardware circuit or a programmed computer. The program may be provided by, for example, a computer-readable recording medium such as a compact disc read only memory (CD-ROM), or may be provided online via a network such as the Internet. In this case, a program recorded in the computer-readable recording medium is usually transferred to and stored in a memory such as a hard disk. In addition, the program may be provided as an independent application software, or may be incorporated into software of the information processing device 2 as a function of the information processing device 2.

A processing unit of the flowchart in the embodiment is divided according to a main processing content in order to facilitate understanding of each processing. The presently disclosed subject matter is not limited by the way of classifying the processing steps. Each processing may be divided into more processing steps. In addition, one processing step may execute more processing.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. An information processing device for processing ultrasonic wave information obtained from an ultrasonic wave sensor including a plurality of ultrasonic wave elements, the information processing device comprising:
an obtainer configured to obtain the ultrasonic wave information from the ultrasonic wave sensor provided on a body surface of a subject;
a recognizer configured to recognize a first blood vessel of the subject and a second blood vessel, based on the ultrasonic wave information, the second blood vessel being different from the first blood vessel; and
a controller configured to cause a display to display comparison information in which first blood vessel information relating to the first blood vessel and second blood vessel information relating to the second blood vessel are compared with each other.

2. The information processing device according to claim 1,
wherein the recognizer is configured to recognize respective positions of the first blood vessel and the second blood vessel.

3. The information processing device according to claim 1,
wherein the comparison information includes information in which the first blood vessel information and the second blood vessel information in the same time period are compared with each other.

4. The information processing device according to claim 1,
wherein the comparison information includes information in which the first blood vessel information and the second blood vessel information in a first time period, and the first blood vessel information and the second blood vessel information in a second time period are compared with each other, the second time period being different from the first time period.

5. The information processing device according to claim 1,
wherein the comparison information includes information in which the first blood vessel information and the second blood vessel information are compared with each other over time.

6. The information processing device according to claim 5,
wherein the comparison information includes information representing respective changes over time in the first blood vessel information and the second blood vessel information.

7. The information processing device according to claim 5,
wherein the comparison information includes information representing a change over time in integrated information, the integrated information being obtained by integrating the first blood vessel information and the second blood vessel information in the same time period.

8. The information processing device according to claim 1,
wherein the comparison information includes at least one of a graph or a numerical value in which the first blood vessel information and the second blood vessel information are compared with each other.

9. The information processing device according to claim 1, further comprising:
a calculator configured to calculate the first blood vessel information and the second blood vessel information, based on the ultrasonic wave information,
wherein the controller is configured to cause the display to display the comparison information in which the first blood vessel information and the second blood vessel information are compared with each other, the first blood vessel information and the second blood vessel information being calculated by the calculator.

10. The information processing device according to claim 1, further comprising:
an event obtainer configured to obtain event information relating to an event occurring in the subject,
wherein the controller is configured to cause the display to display the event information together with the comparison information.

11. The information processing device according to claim 10,
wherein the event information includes medication event information relating to medication to the subject.

12. The information processing device according to claim 11,
wherein the medication event information includes information relating to at least one of a type, a dose, or an administration time of a drug administered to the subject.

13. The information processing device according to claim 1,
wherein the first blood vessel information includes information relating to at least one of a flow velocity or a flow rate of the first blood vessel, and
the second blood vessel information includes information relating to at least one of a flow velocity or a flow rate of the second blood vessel.

14. The information processing device according to claim 1,
wherein the first blood vessel and the second blood vessel are branched from a third blood vessel at a proximal position.

15. The information processing device according to claim 1,
wherein the first blood vessel and the second blood vessel are an internal carotid artery and an external carotid artery.

16. An information processing system comprising:
an ultrasonic wave sensor including a plurality of ultrasonic wave elements; and
the information processing device according to claim 1.

17. The information processing system according to claim 16,
wherein the ultrasonic wave sensor further includes a sheet-shaped member attached to a body surface of a subject.

18. A non-transitory computer readable storage medium storing an information processing program for processing ultrasonic wave information obtained from an ultrasonic wave sensor including a plurality of ultrasonic wave elements,
the information processing program comprising instructions which, when executed by a computer, causes the computer to:
obtain the ultrasonic wave information from the ultrasonic wave sensor provided on a body surface of a subject;
recognize a first blood vessel of the subject and a second blood vessel, based on the ultrasonic wave information, the second blood vessel being different from the first blood vessel; and
cause a display to display comparison information in which first blood vessel information relating to the first blood vessel and second blood vessel information relating to the second blood vessel are compared with each other.
